(19) United States
(12) Patent Application Publication
(10) Pub. No.: US 2025/0198916 A1
(43) Pub. Date: Jun. 19, 2025

(54) SYSTEM AND APPARATUS FOR FLUID MONITORING

(71) Applicant: WaterSight Ltd., Ness Ziona (IL)

(72) Inventors: Eli ASSOOLIN, Ness Ziona (IL); Eyal YATSKAN, Ness Ziona (IL); Lital COHEN, Ness Ziona (IL)

(21) Appl. No.: 18/845,750

(22) PCT Filed: Mar. 9, 2023
(86) PCT No.: PCT/IL2023/050250

§ 371 Date: Sep. 10, 2024

Related U.S. Application Data
(60) Provisional application No. 63/269,112, filed on Mar. 10, 2022.

(51) Int. Cl. G01N 21/3577; G01N 21/359; G01N 33/18

(52) U.S. Cl. CPC G01N 21/3577; G01N 21/359; G01N 33/1893

(57) ABSTRACT

An apparatus for fluid monitoring is provided. The apparatus includes a fluid conduit having an inlet and an outlet, the fluid conduit includes a transparent portion. The apparatus further includes a light source configured to emit light through the transparent portion and to illuminate the fluid inside the fluid conduit with the light, and a detector configured for detecting light transmitted through or reflected from the fluid. The detector including a pixel array having a plurality of pixels each of which being configured to detect intensity of one wavelength within a spectrum of the light such that the pixel array obtains a spectral signature of the fluid including intensities of wavelengths within the spectrum.

### FIELD OF INVENTION

The presently disclosed subject matter relates to an apparatus for fluid monitoring, in general, and in particular to an apparatus for an inline water monitoring.

### BACKGROUND

Measuring and maintaining the quality of water is important in a wide variety of circumstances. For example, for keeping fish and/or other aquatic life, the quality of the water must be kept within certain tolerances to keep the aquatic life healthy. As another example, the water in swimming and diving pools, hot tubs, and other sports, recreational, and therapeutic bodies of water need to be kept at certain levels of quality not only to maintain that water's clarity, but also to keep the users of these bodies of water safe from waterborne illnesses. As yet another example, the quality of potable water needs to be maintained within a range of tolerances as to a variety of chemical constituents for any one or more of a number of reasons, such as to make the water safe for ingesting, less harmful to distribution systems, and to promote healthfulness of the drinkers.

### SUMMARY OF INVENTION

There is provided, according to one aspect of the presently disclosed subject matter an apparatus for fluid monitoring. The apparatus includes a fluid conduit having an inlet and an outlet, the fluid conduit includes a transparent portion. The apparatus further includes a light source configured to emit light through the transparent portion and to illuminate the fluid inside the fluid conduit with the light, and a detector configured for detecting light transmitted through or reflected from the fluid. The detector including a pixel array having a plurality of pixels each of which being configured to detect intensity of one wavelength within a spectrum of the light such that the pixel array obtains a spectral signature of the fluid including intensities of wavelengths within the spectrum.

The apparatus can further include a housing configured for holding the fluid conduit and having an inlet coupling member in fluid communication with the inlet and an outlet coupling member in fluid communication with the outlet, the inlet and outlet coupling members are configured for coupling to pipe elements in a fluid pipeline.

The optical system can include a seat configured to hold the fluid conduit, wherein the light source is mounted on a first side of the seat and the detector is mounted on a second side of the seat, such that an optical path is formed between the light source and the detector through the transparent portion.

The array of pixels can be arranged along the length of the detector, such that each of the pixels is configured to detect a certain wavelength within the spectrum, such that the entire array of pixels is configured to provide information regarding each wavelength within the spectrum.

The detector can include a band pass filter disposed along the array of pixels and being configured to filter various wavelengths of the spectrum such that each of the pixels on the array of pixels receives light of a certain wavelength or bandwidth.

The detector can be provided with a linear filter configured such that each location along a first dimension of the linear filter allows transmitting light of a single wavelength or a narrow bandwidth of wavelengths.

The apparatus can further include an optical guiding member for directing illumination from the light source to the transparent portion and being configured to form an even and orthogonal illumination, such that the fluid inside the fluid conduit is evenly illuminated.

The optical guiding member can include an array of blocking walls each having an elongated slit extending along length of the transparent portion such that light arrays which are not directed orthogonally to the transparent portion are blocked by one of the blocking walls.

The detector can be configured to detect light intensity in wavelengths that range between 400 nm and 1100 nm and provides 1024 with up-to 12 bit digital values.

The apparatus can further include a controller configured for analyzing a spectral signature of the fluid flowing through the fluid conduit, the controller is configured for obtaining the spectral signature and for extracting characterizing features of the spectral signature.

The characterizing features are light properties, of predetermined wavelengths in the illuminated spectrum.

The controller can be configured for comparing the characterizing features with corresponding features stored in a database.

There is provided according to one aspect of the presently disclosed subject matter a system for fluid monitoring, the system includes at least one apparatus mounted in a fluid pipeline, the apparatus includes:
a fluid conduit having an inlet and an outlet coupled to the pipeline, the fluid conduit includes a transparent portion;
a light source configured to emit light through the transparent portion and to illuminate the fluid inside the fluid conduit with the light;
an optical system including detector configured for detecting light transmitted through or reflected from the fluid, the detector including a pixel array having a plurality of pixels each of which being configured to detect intensity of one wavelength within a spectrum of the light such that the pixel array obtains a spectral signature of the fluid including intensities of wavelengths within the spectrum
a wireless transmitting module for transmitting data obtained by the optical system;

The system further includes a server for collecting the spectral signature from the at least one apparatus and for determining quality of fluid in the pipeline.

The server can be configured for classification of the spectral signature in accordance with the prestored spectral signatures, wherein each one of the prestored spectral signatures is associated with at least one property of the fluid.

The server can be configured for classification of the spectral signature in accordance with location of the at least one apparatus along the pipeline.

The apparatus can be coupled to the pipeline with an inlet connector and an outlet connector configured to eliminate air in the fluid conduit.

The inlet connector can be coupled to the inlet of the fluid conduit and the outlet connector is coupled to the outlet, the inlet connector is configured to urge fluid into the fluid conduit at a pressure higher than the pressure at the outlet.

The detector can include at least one natural pixel configured to detect any light in the range of 400 to 1100 nm, and wherein the optical system is configured to send an alert when light detected by the at least one natural pixel is below a predetermined threshold.

The server can be configured to send the apparatus instructions related to the spectral signature.

### BRIEF DESCRIPTION OF THE DRAWINGS

In order to understand the disclosure and to see how it may be carried out in practice, embodiments will now be described, by way of non-limiting examples only, with reference to the accompanying drawings, in which:

FIG. 1A is a side perspective view of an apparatus for fluid monitoring;

FIG. 1B is a side perspective view of the apparatus of FIG. 1A with the covers thereof removed;

FIG. 2 is an exploded view of the apparatus of FIG. 1A;

FIG. 3A is a perspective view of the optical system of the apparatus of FIG. 1A in accordance with an example of the presently disclosed subject matter;

FIG. 3B is a left side view of the optical system of the apparatus of FIG. 1A;

FIG. 4 is an exploded view of the optical system of FIG. 3A; and

FIG. 5 is a side view of the holder of the optical system of FIG. 3A.

### DETAILED DESCRIPTION OF EMBODIMENTS

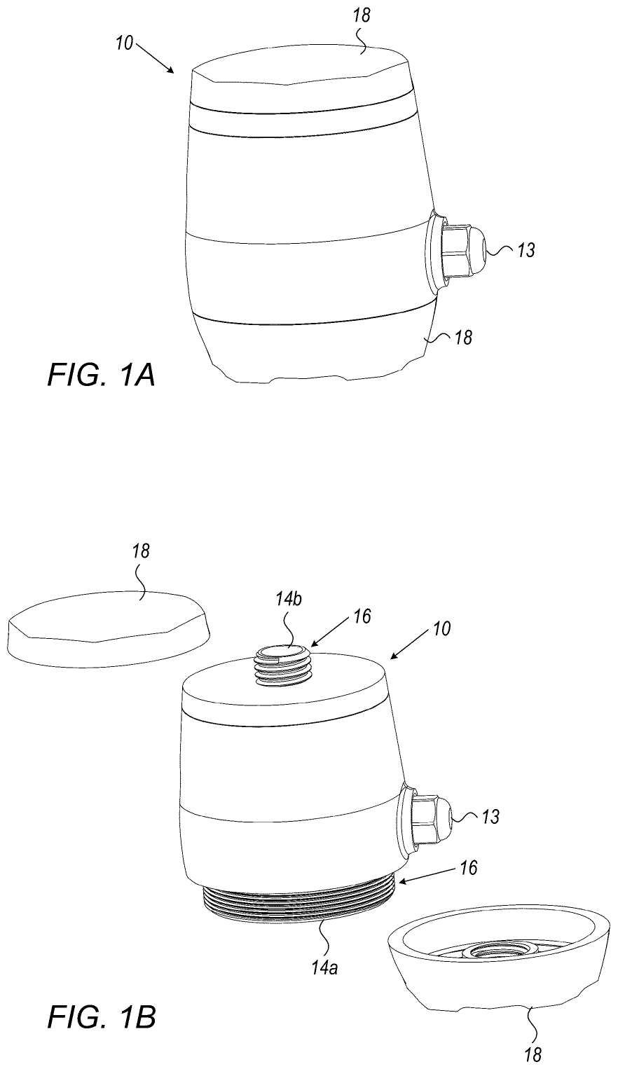

As shown in FIGS. 1A and 1B, the apparatus 10 for fluid monitoring can include a housing 12 having an inlet coupling member 14a and an outlet coupling member 14b, each of which is configured for coupling to pipe elements. For example, the inlet and outlet coupling members 14a and 14b can include screw threads 16, for coupling to other pipe elements having corresponding screw threads. This way, the apparatus 10 can be mounted in a pipeline in which fluid is transported, for example, water pipeline, thereby allowing real time monitoring of the water. As shown, the inlet and outlet coupling members 14a and 14b can be provided with corresponding covers 18, for protecting the apparatus 10, while not coupled to a pipeline. The apparatus 10 can further include a wire coupling member 13, as explained hereinafter.

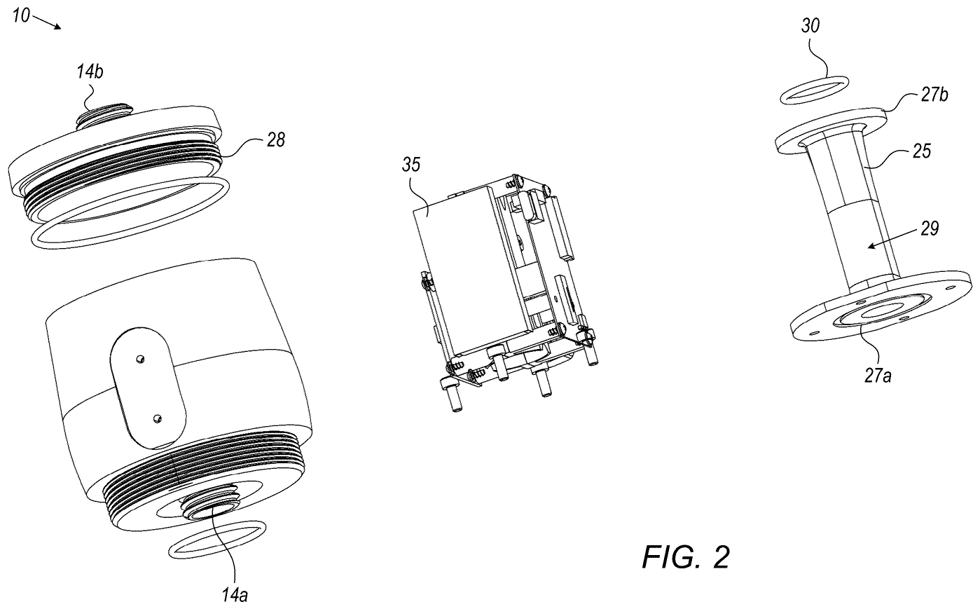

With reference to FIG. 2, the apparatus 10 further includes a fluid conduit 25 and an optical system 35 mounted inside the housing 12. The fluid conduit 25 has an inlet 27a and an outlet 27b, which are configured to be coupled to the inlet and outlet coupling members 14a and 14b, respectively. For example, the fluid conduit 25 can be disposed inside the housing 12, such that the inlet 27a engages an inner surface of the inlet coupling member 14a. The inlet 27a can be affixed to the inlet coupling member 14a by screws or other coupling means. Similarly, the outlet 27b engages an inner surface of the outlet coupling member 14b. Seal members 30 can be provided to ensure watertight coupling of fluid conduit 25 to the inlet and outlet coupling members 14a and 14b.

According to the present example, the outlet coupling member 14b is removably coupled to the housing 12 by a screw thread 28, allowing the opening housing 12 and accessing the fluid conduit 25 and the optical system 35 inside the housing 12. This allows cleaning and maintenance of the optical system 35. The fluid conduit 25 includes a transparent portion 29, allowing light to illuminate the fluid inside the fluid conduit 25 and is disposed inside the housing 12 adjacent to the optical system 35 such that the light illuminated through the transparent portion 29 can be assessed by the optical system 35.

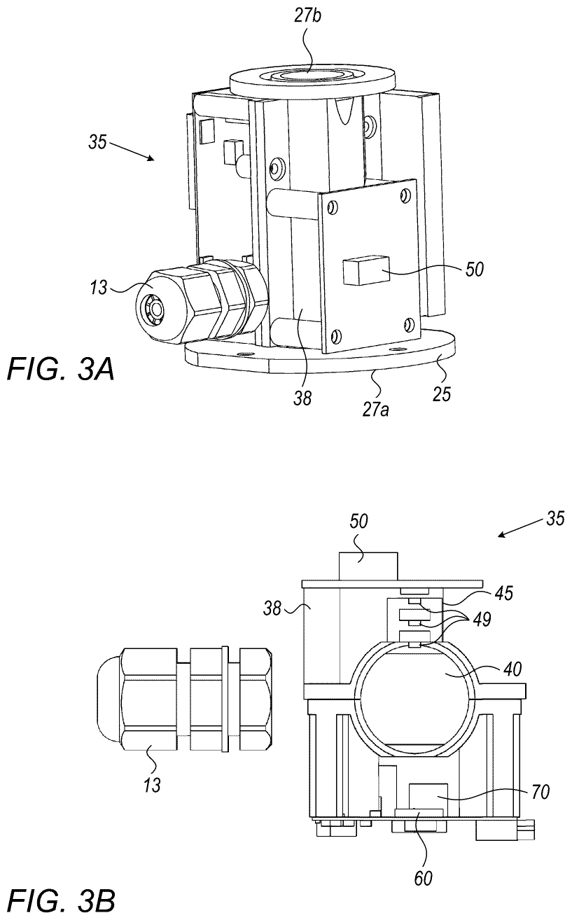

According to an example, as shown in FIGS. 3A and 3B, the optical system 35 includes a holder 38 configured to hold the fluid conduit 25. The holder 38 is provided with a seat 40 having a shape corresponding to the shape of the fluid conduit 25. For example, the fluid conduit 25 can be a cylindrical member and the seat 40 can have a corresponding cylindrical shape such that the fluid conduit 25 snugly fits in the seat.

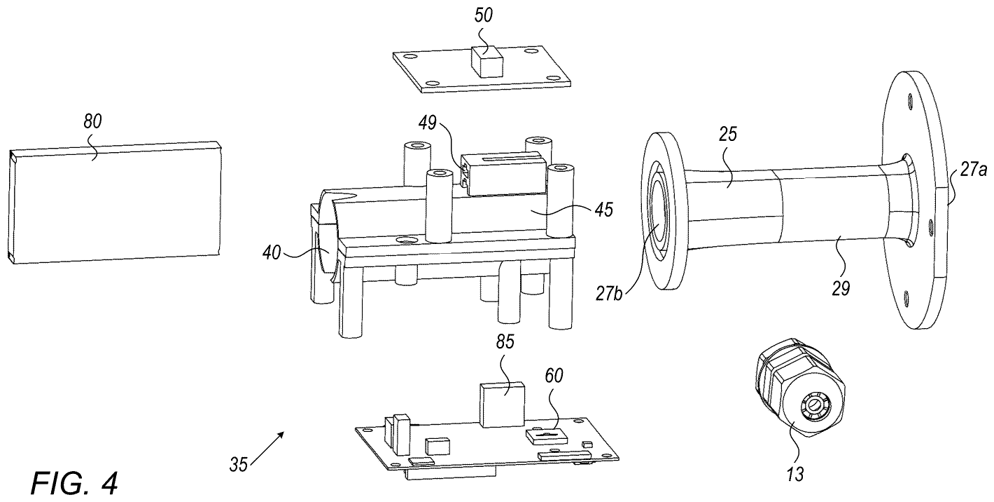

As shown in FIGS. 3A-3B and 4, the optical system 35 includes a light source 50 disposed on a first side of the seat 40 and a detector 60 disposed on a second side of the seat 40, such that an optical path is formed between the light source 50 and the detector 60. More particularly, the light source 50 and the detector 60 are disposed such that the transparent portion 29 of the fluid conduit 25 is disposed along the same optical path. This way, light from the light source 50 illuminates the fluid inside the fluid conduit 25 through the transparent portion 29 the light then travels towards the detector 60 on the other side of the fluid conduit 25. It is appreciated that according to this example, the transparent portion 29 extends along at least a portion of the circumference of the fluid conduit 25, such that light from the light source 50 can enter the fluid conduit 25 and at the same time the light can then travel towards the detector 60.

According to another example, the light source 50 and the detector 60 can be disposed at the same side of the transparent portion 29. According to this example, the detector 60 can be configured to detect light reflected from the fluid, as opposed to light transmitting through the fluid.

The light source 50 is configured to illuminate the fluid inside the fluid conduit 25 with light of a predetermined spectrum, the detector 60 on the other hand, is configured to detect wavelengths within the illuminated spectrum.

According to an example, the detector 60 includes an array of pixels arranged along the length of the detector 60, each of the pixels is configured to detect a certain wavelength within the spectrum, such that the entire array of pixels is configured to provide information regarding each wavelength within the spectrum.

According to the illustrated example, the detector 60 includes a band pass filter 70, such as a linear variable filter, disposed along the array of pixels and being configured to filter various wavelengths of the spectrum. The filter 70 is configured such that each of the pixels on the array of pixels receives light of a certain wavelength or bandwidth. This way, each pixel provides information regarding parameters of light within a specific wavelength, and the detector 60 provides information regarding each of the wavelengths within the illuminating spectrum.

Consequently, the apparatus 10 allows illuminating fluid inside the fluid conduit 25 with light of a predetermined spectrum and obtain information regarding light absorbance of each of the wavelengths within the illuminated spectrum.

According to another example, each of the pixels in the detector 60 can be provided with a designated filter, such that each pixel receives light of a predetermined wavelength. The pixel array of the detector 60 can be for example as describe in U.S. patent application Ser. No. 16/462,760 “ACTIVE-PIXEL SENSOR ARRAY”, the disclosure of which is incorporated herein by reference.

The detector 60 is thus configured for detecting light of a wide spectrum transmitted through the fluid, and each pixels is configured to detect the intensity of one of the wavelength within the spectrum. The detector thus obtains a spectral signature of the fluid, including intensities of wavelengths within the spectrum. The spectral signature, i.e. the light absorption of the fluid in each wavelength, can be used to provide information regarding substances of the fluid and facilitate detecting the quality of the fluid, such as detecting water containment.

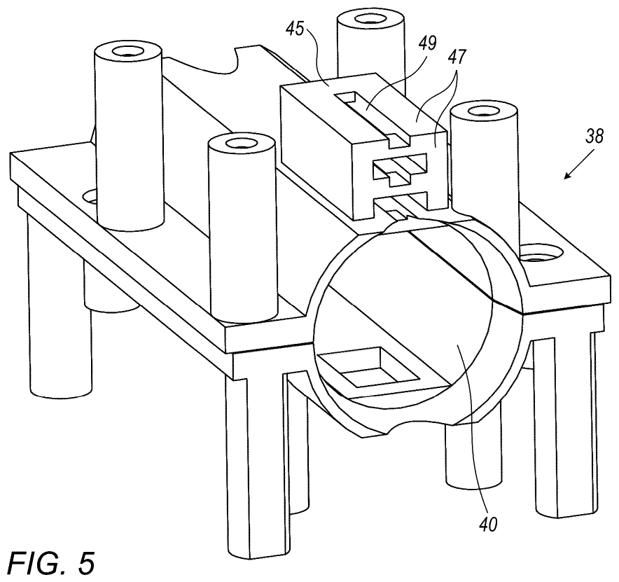

As shown in FIG. 5, the holder 38 of the optical system 35 can further include an optical guiding member 45 for directing the illumination from the light source to the seat 40 and the fluid conduit 25. The optical guiding member 45 is configured to form an even and orthogonal illumination, such that the transparent portion 29 is evenly illuminated, and reflections are precluded. According to the illustrated example, the optical guiding member 45 includes an array of blocking walls 47 each having an elongated slit 49, extending along the length of the transparent portion 29. This way, light arrays that are not directed orthogonally to the transparent portion 29 are blocked by one of the blocking walls 47. The optical guiding member 45 thus provides an evenly distributed illumination along the transparent portion 29.

According to an example, the detector 60 can be configured to detect light intensity in wavelengths that range between 400 nm and 1100 nm and provides 1024 with up-to 12 bit digital values. Each value represents the intensity of each wavelength. These 1024 digital value vectors allow the creation of a high-resolution spectral signature of any tested substance in the range of 400 to 1100 nm.

The pixel array of the detector 60 can be for example as describe in PCT Patent application published as WO/2021/224900-DEVICE AND METHOD FOR SPECTRAL ANALYSIS OF A COMPOUND SPECIMEN. Specifically, as described with respect to FIGS. 4A to 9B, the disclosure of which is incorporated herein by reference. It would be appreciated that while the device described in this patent application provides a detector for detecting light in the range of 400 to 700 nm, such a detector can be modified to detect light in the range of range of 400 to 1100 nm, as disclosed in the present application. The use of such detector allows for obtaining spectral signatures including the visible spectrum as well as the near infrared spectrum.

As indicated above, the apparatus 10 can further include a wire coupling member 13, allowing extending wires from the optical system 35 out of the apparatus 10. For example, for providing the optical system 35 with electrical power or for extracting data accumulated by the detector 60. The apparatus 10 can further include wireless transmitting module 80, allowing wirelessly transmitting data obtained by the detector 60. Module 80 can further be configured to receive data related to the desired spectrum, or wavelength to be detected.

According to an example, the apparatus 10 further include a controller 85 configured for analyzing a spectral signature of the fluid. The controller 85 can be configured for obtaining a spectral signature of the fluid, and for extracting characterizing features of the spectral signature. The characterizing features can be light properties, such as absorbance, of predetermined wavelengths in the illuminated spectrum. The controller can be further configured for comparing the characterizing features with corresponding features stored in a database. The corresponding features include light properties of the predetermined wavelengths of a fluid, including a predetermined substance. For example, the corresponding features can include light absorbance of a certain wavelength of water containing a certain containment.

Accordingly, the module 80 can be configured to receive instructions regarding desired characterizing features to be detected. For example, if a certain containment is suspected to be found in the water, a set of instructions can be sent from a remote server. This way, the apparatus 10 can be dynamically controlled to obtain spectral signature specific to predetermined substances in the water. The apparatus 10 can further be configured for self-learning of the quality of the fluid. For example, it would be appreciated that spectral signatures of the same fluid may differ depending on the location along the pipeline where the apparatus is installed. However, since the apparatus is installed as an inline element, continuous detection of the quality of the fluid can be obtained. This way, the apparatus 10 can be configured to obtain a baseline of an expected spectral signature. In other words, the apparatus 10 can be configured to determine the usual spectral signature of the water running through the fluid conduit 25 and to determine deviation from the expected spectral signature for the specific location.

Moreover, according to an aspect of the invention, a water monitoring system can include a plurality of apparatuses 10 mounted at different locations along a pipeline and a remote server configured to receive data from each of apparatuses 10. The system can be configured to allow the classification of various spectral signatures. For example, when an abnormal spectral signature is detected, an independent water quality test can be conducted to determine the substance that affected the spectral signature. Data related to the findings of the water quality test can be stored in the server in association with the abnormal spectral signature. For example, in case the water quality test determines the existence of high iron level, the spectral signature which was obtained can be stored at the server in association with the specific iron level. This way, the server can built overtime a database of various spectral signatures, each of which being associated with a specific type of water containment.

Thus, the system can be configured not only to detect water containment but also to indicate the specific substance in the water and the level of such substance. It would be appreciated determining the substance and its level can be carried out by implementing various classification methods, for example, as described in international application WO2021229555 “A METHOD AND A SYSTEM FOR ANALYZING A SPECTRAL SIGNATURE OF A COMPOUND SPECIMEN”. Since the detector 60 is configured to provide a high-resolution spectral signature of any tested substance in the range of 400 to 1100 nm, the system can collect enormous amount of spectral signatures each of which being associated with a specific properties of the water including temperature, acidity (pH), dissolved solids, particulate matter, dissolved oxygen, hardness and suspended sediment etc.

It would be appreciated that spectral signatures can be affected by various other conditions, such as a specific location along of the apparatus along the pipeline. In other words, water at a first location along the pipeline may have a certain spectral signature, while water at a second location a pipeline may have another certain spectral. This may be caused, by varying temperatures or other conditions. Hence, the system can be configured to determine a spectral signature which is served as a baseline for each apparatus installed along the pipeline. This way, the system can learn the normal condition for the specific location, for example, by condition based monitoring, while constantly taking spectral images and broadcasting them to the server for data analysis. Once an anomaly is detected in one of the apparatuses, an alert notification will be generated for the specific apparatus.

It is appreciated that since the apparatus 10 is configured for obtaining spectral signature of flowing fluids, it is required to ensure that the fluid is homogeneous, such that the moving particles do not affect the spectral signature. Specifically, small bubbles such as dissolved air, may cause variations in the spectral signature due to differences between the light absorbance of the air and that of the fluid. Hence, in order to eliminate or at least minimize the amount of air inside the fluid conduit 25, the apparatus can be provided with connectors configured to eliminate air from entering the apparatus 10. The connectors can be configured to couple the apparatus 10 to a pipeline, such that the flow rate at the inlet coupling member 14a is low. The optical system 35 can be configured for capturing spectral signatures at a high rate, for example 4000 per second. In other words, the optical system 35 can be configured to obtain spectral signatures at a rate that is determined in accordance with the flow rate, such that the variations in light absorbance caused by the movement of the fluid is reduced.

According to an example, an inlet connector can be coupled to the inlet coupling member 14a and an outlet connector can be coupled to the outlet coupling member 14b. The inlet and outlet connectors can be configured such that fluid entering the inlet coupling member 14a is at a pressure higher than the pressure at the outlet coupling member 14b. This way, the fluid conduit 25 will be completely occupied with fluid ensuring thereby that the light absorbance is homogeneous. According to an example, the pressure at the inlet and outlet connectors can be configured such that the pressure of fluid entering the inlet coupling member 14a is 1 bar, while pressure at the outlet coupling member 14b is 0.5 bar.

As indicated above and as described in Patent application published as WO/2021/224900-DEVICE AND METHOD FOR SPECTRAL ANALYSIS OF A COMPOUND SPECIMEN, the detector 60 includes a pixel array each of which being configured to detect a certain wavelength in the range of 400 to 1100 nm. According to an example, the detector 60 can include some natural pixels which are configured to detect any light in the range of 400 to 1100 nm. In other words, while most of the pixels in the detector 60 are configured to detect only light at a specific wavelength, the natural pixels are sensitive to any light in the range of 400 to 1100 nm. These natural pixels can be configured to detect dirt that may block the detector 60 from properly obtaining a spectral signature.

Thus, when the amount of light detected by the natural pixels is below a predetermined threshold, the apparatus 10 can be configured to send an alert, indicating the fluid conduit 25 has to be cleaned so as to allow obtaining an accurate spectral signature. Moreover, the system can be configured to determine the threshold of the natural pixels, i.e., the threshold at which spectral signature can no longer be obtained. This way, cleaning of the apparatus 10 is carried out only when the required spectral signature can no longer be obtained.

Furthermore, since dirt may appear in the water at various times of the day, the apparatus 10 can further be configured such that detection of the predetermined threshold is classified in accordance with the time of the day. This way, when the light detected by the natural pixels is below the predetermined threshold, the system can determine whether or not the low light detection is caused by a temporal change in the color of the water or dirt which is accumulated over the fluid conduit 25.

According to an example, the system can further include a docking station which is configured for readout of the apparatus 10. In other words, the docking station can be configured with a power source and a communication module, such that the docking station receives data from the apparatus 10 and sends the data to the remote server. The docking station thus facilitates communication with the apparatus 10.

Those skilled in the art to which the presently disclosed subject matter pertains will readily appreciate that numerous changes, variations, and modifications can be made without departing from the scope of the invention, mutatis mutandis.

1. An apparatus for fluid monitoring comprising:
a fluid conduit having an inlet and an outlet, said fluid conduit includes a transparent portion;
a light source configured to emit light through said transparent portion and to illuminate the fluid inside the fluid conduit with said light;
an optical system including detector configured for detecting light transmitted through or reflected from said fluid, said detector including a pixel array having a plurality of pixels each of which being configured to detect intensity of one wavelength within a spectrum of said light such that said pixel array obtains a spectral signature of said fluid including intensities of wavelengths within said spectrum.

2. The apparatus according to claim 1 further comprising a housing configured for holding said fluid conduit and having an inlet coupling member in fluid communication with said inlet and an outlet coupling member in fluid communication with said outlet, said inlet and outlet coupling members are configured for coupling to pipe elements in a fluid pipeline.

3. The apparatus according to claim 1 wherein said optical system includes a seat configured to hold the fluid conduit, wherein said light source is mounted on a first side of the seat and said detector is mounted on a second side of the seat, such that an optical path is formed between the light source and the detector through said transparent portion.

4. The apparatus of claim 1 wherein the array of pixels is arranged along the length of the detector, such that each of the pixels is configured to detect a certain wavelength within the spectrum, such that the entire array of pixels is configured to provide information regarding each wavelength within the spectrum.

5. The apparatus of claim 4 wherein the detector includes a band pass filter disposed along the array of pixels and being configured to filter various wavelengths of the spectrum such that each of the pixels on the array of pixels receives light of a certain wavelength or bandwidth.

6. The apparatus of claim 5 wherein said detector is provided with a linear filter configured such that each location along a first dimension of the linear filter allows transmitting light of a single wavelength or a narrow bandwidth of wavelengths.

7. The apparatus of claim 6 further comprising an optical guiding member for directing illumination from the light source to the transparent portion and being configured to form an even and orthogonal illumination, such that the fluid inside the fluid conduit is evenly illuminated.

8. The apparatus of claim 7 wherein the optical guiding member includes an array of blocking walls each having an elongated slit extending along length of the transparent portion such that light arrays which are not directed orthogonally to the transparent portion are blocked by one of the blocking walls.

9. The apparatus of claim 1 wherein said detector is configured to detect light intensity in wavelengths that range between 400 nm and 1100 nm and provides 1024 with up-to 12 bit digital values.

10. The apparatus of claim 1 further comprising a controller configured for analyzing a spectral signature of the fluid flowing through the fluid conduit, said controller is configured for obtaining the spectral signature and for extracting characterizing features of the spectral signature.

11. The apparatus of claim 10 wherein the characterizing features are light properties, of predetermined wavelengths in the illuminated spectrum.

12. The apparatus of claim 11 wherein the controller is configured for comparing the characterizing features with corresponding features stored in a database.

13. A system for fluid monitoring, the system comprising:
at least one apparatus mounted in a fluid pipeline, said apparatus includes:
a fluid conduit having an inlet and an outlet coupled to said pipeline, said fluid conduit includes a transparent portion;
a light source configured to emit light through said transparent portion and to illuminate the fluid inside the fluid conduit with said light;
an optical system including detector configured for detecting light transmitted through or reflected from said fluid, said detector including a pixel array having a plurality of pixels each of which being configured to detect intensity of one wavelength within a spectrum of said light such that said pixel array obtains a spectral signature of said fluid including intensities of wavelengths within said spectrum
a wireless transmitting module for transmitting data obtained by the optical system;
a server for collecting said spectral signature from said at least one apparatus and for determining quality of fluid in said pipeline.

14. The system according to claim 13 wherein said server is further configured for classification of said spectral signature in accordance with the prestored spectral signatures, wherein each one of said prestored spectral signatures is associated with at least one property of said fluid.

15. The system according to claim 14 wherein said server is further configured for classification of said spectral signature in accordance with location of said at least one apparatus along said pipeline.

16. The system according to claim 13 wherein said apparatus is coupled to said pipeline with an inlet connector and an outlet connector configured to eliminate air in said fluid conduit.

17. The system according to claim 16 wherein said inlet connector is coupled to said inlet of said fluid conduit and said outlet connector is coupled to said outlet, said inlet connector is configured to urge fluid into said fluid conduit at a pressure higher than the pressure at said outlet.

18. The system according to claim 13 wherein said detector includes at least one natural pixel configured to detect any light in the range of 400 to 1100 nm, and wherein said optical system is configured to send an alert when light detected by said at least one natural pixel is below a predetermined threshold.

19. The system according to claim 13 wherein said server is configured to send said apparatus instructions related to said spectral signature.
